(19) United States
(12) Patent Application Publication
(10) Pub. No.: US 2011/0183944 A1
(43) Pub. Date: Jul. 28, 2011

(54) SUSTAINED-RELEASE NSAID/HMG CoA REDUCTASE INHIBITOR COMPOSITIONS

(76) Inventors: Paul Ashton, Newton, MA (US); Hong Guo, Wayland, MA (US); Jianbing Chen, Wayland, MA (US); Tadeusz Cynkowski, Brookline, MA (US); Grazyna Cynkowska, Brookline, MA (US)

(21) Appl. No.: 13/015,058

(22) Filed: Jan. 27, 2011

Related U.S. Application Data
(60) Provisional application No. 61/299,172, filed on Jan. 28, 2010.

(51) Int. Cl. A61K 31/635; A61P 25/16; A61P 25/28; A61P 35/00

(52) U.S. Cl. 514/158

(57) ABSTRACT

Compositions for controlled release of one or more therapeutic agents where the composition is essentially free of excipients are disclosed. In particular, compositions comprising a HMG-CoA reductase inhibitor, particularly simvastatin, and a NSAID, such as a COX-2 inhibitor, particularly celecoxib, in which greater than 90% of the weight of the composition is made up of the HMG-CoA reductase inhibitor and NSAID are provided.

### RELATED APPLICATIONS

This application claims the benefit of U.S. Provisional Application No. 61/299,172, filed Jan. 28, 2010, the contents of which are incorporated by reference in their entirety.

### BACKGROUND OF THE INVENTION

There are many useful drugs on the market today for which traditional means of administration are far from ideal. Bolus injections and oral unit doses typically result in a high initial systemic concentration of the active agent, in excess of the therapeutic concentration, which falls off over time and which will fall below the therapeutic concentration if another bolus is not timely administered. The result is that the ideal therapeutic concentration is not consistently maintained, there is a risk of toxicity associated with high systemic exposure to the drug, and the maintenance of a minimally effective concentration is dependent upon repeated administration at prescribed intervals. Patient compliance with a dosing regimen is difficult to ensure, especially where the course of therapy is long or of indeterminate or lifetime duration. There is a need for methods to deliver these drugs more effectively, so that therapeutic concentrations are maintained constantly in the tissues intended to be treated over an extended period of time, with minimal vulnerability to the vagaries of patient compliance, and ideally with minimal systemic exposure or exposure of uninvolved tissues and organs.

Extended-release and controlled-release drug delivery systems have been developed to address these needs. Implanted pumps and reservoirs, with various mechanisms for regulating release of drugs, were among the first solutions to be developed. A wide variety of polymeric matrices, permeated with drug substance, have also been developed which serve as implantable drug reservoirs. These polymeric implants gradually release drug over the course of days, weeks, or months as the contained drug diffuses through and out of the matrix and into the surrounding tissue. Three principal advantages provided by polymeric drug delivery compositions are:

(1) Localized delivery of drug. The product can be implanted directly at the site where drug action is needed and hence systemic exposure of the drug can be reduced. This becomes especially important for toxic drugs which are related to various systemic side effects (such as chemotherapeutic drugs).

(2) Sustained delivery of drug. The drug is released over extended periods, eliminating the need for multiple injections or oral doses. This improves patient compliance, especially for drugs for chronic indications requiring frequent administration, such as replacement therapy for enzyme or hormone deficiencies, or for extended antibiotic treatments for such tenacious diseases as tuberculosis.

(3) Stabilization of the drug. The polymer matrix protects the drug from the physiological environment, particularly circulating enzymes, thereby improving stability in vivo. This makes the technology particularly attractive for the delivery of labile proteins and peptides.

For the reasons above, the use of drug-infused polymer implants as sustained-release drug delivery devices is now well established. One class of existing implants consists of preformed devices, ranging in size from matchstick-sized cylindrical rods such as the Norplant™ (levonorgestrel) and Zoladex™ (goserelin acetate) implants, to microspheres such as are sold under the trade name Lupron Depot™ (leuprolide acetate).

A major disadvantage of the macroscopic devices is their physical size. Implantation of Zoladex™ rods, for instance, requires the use of 14- or 16-gauge needles, and implantation of Norplant™ rods requires a surgical incision under local anesthesia, with similar subsequent procedures to replace and/or remove them.

Micro- or macroscopic implants that are capable of releasing a therapeutic agent in a sustained fashion and do not require surgical removal following delivery of the agent would be of great value in the field of drug delivery.

### SUMMARY OF THE INVENTION

The invention provides compositions comprising a 3-hydroxy-3-methyl-glutaryl-coenzyme A (HMG-CoA) reductase inhibitor, particularly simvastatin, and a non-steroidal anti-inflammatory drug (NSAID), such as a cyclooxygenase-2 (COX-2) inhibitor, particularly celecoxib, in which greater than 90% of the weight of the composition is made up of the HMG-CoA reductase inhibitor and NSAID. In certain embodiments, compositions of the invention may have an excess of one of simvastatin or celecoxib with respect to the other, e.g., the mole ratio of celecoxib to simvastatin is selected from 3:1 to 1:3, e.g., 2:1, 2.75:1, 1:2 or 1:2.75. In certain embodiments, greater than 95% of the weight of the composition is celecoxib and simvastatin, such as greater than 99%. In certain embodiments, the composition is essentially free of excipients and diluents. In certain embodiments, the composition provides sustained release of simvastatin and/or celecoxib in a biological medium over a period of hours, days or weeks.

The invention further provides methods of preparing a drug delivery composition of simvastatin and celecoxib, comprising: a. providing a solution comprising a water-miscible solvent, simvastatin, and celecoxib, and b. contacting the solution with an aqueous medium under conditions that induce precipitation of a solid comprising simvastatin and celecoxib. In certain embodiments, the method further comprises isolating the solid from the aqueous medium.

The invention also provides a method for treating or preventing a disease, e.g., neurodegenerative disease or disorder, comprising administering a drug delivery composition or formulation of the invention to a patient in need of treatment.

### BRIEF DESCRIPTION OF DRAWINGS

FIG. 1. Release profile of celecoxib and simvastatin from celecoxib/simvastatin precipitate.

### DETAILED DESCRIPTION OF THE INVENTION

### Overview

The present invention provides compositions for controlled release of one or more therapeutic agents where the composition is essentially free of excipients. In particular, the invention provides compositions comprising a HMG-CoA reductase inhibitor, particularly simvastatin, and a NSAID, such as a COX-2 inhibitor, particularly celecoxib, in which greater than 90% of the weight of the composition is made up of the HMG-CoA reductase inhibitor and NSAID. The controlled-release compositions may provide sustained-release drug delivery for one or more therapeutic components of the composition, e.g., sustained release of a NSAID, sustained release of the HMG-CoA reductase inhibitor or sustained release of both the NSAID and HMG-CoA reductase inhibitor. In certain embodiments, the invention comprises methods of treating or preventing a condition in a patient with compositions of the invention where the condition is selected from a neurological condition or cancer. In certain embodiments, the compositions of the invention are administered to a patient by any acceptable method for local or systemic administration such as parenterally, e.g., intravenously, intramuscularly or intrathecally. In certain embodiments, the compositions may be targeted to a particular location within the body such as within or near a tumor. The compositions release one or more of the therapeutic components in a controlled fashion over a period of hours, days, weeks or months.

The invention provides methods of preparing compositions comprising a HMG-CoA reductase inhibitor and a NSAID. In certain embodiments, the method comprises forming or obtaining a solution of two such therapeutic agents, e.g., simvastatin and celecoxib, in a water-miscible organic solvent, e.g., N,N-dimethylformamide, dimethylsulfoxide, or N-methyl-2-pyrrolidone. In certain embodiments, the solution is contacted with an aqueous medium, for example, the solution is introduced in small aliquots, e.g., dropwise, to an aqueous medium, e.g., water, an aqueous solution, aqueous buffer, or a biological system. A solid comprising the HMG-CoA reductase inhibitor and NSAID precipitates from the solution in the form of, for example, micro- or macroscopic particles. In particular embodiments, the aqueous medium is biological, such as within the body of a patient.

In certain embodiments, compositions of the invention may be prepared as pharmaceutical compositions for administration to patients in need thereof. In certain embodiments, compositions of the invention may be used in the treatment or prevention of neurodegenerative disorders such as Alzheimer's disease, Parkinson's disease and Huntington's disease. In particular, compositions of the invention may be useful in reducing oxidative stress and neurological damage. In certain embodiments, compositions of the invention may be used in the treatment of cancer.

### Definitions

For convenience, before further description of the present invention, certain terms employed in the specification, examples, and appended claims are collected here. These definitions should be read in light of the remainder of the disclosure and understood as by a person of skill in the art.

The articles “a” and “an” are used herein to refer to one or to more than one (i.e., to at least one) of the grammatical object of the article. For example, ‘an element’ means one or more than one element.

The terms “biocompatible polymer” and “biocompatibility” when used in relation to polymers are art-recognized. For example, biocompatible polymers include polymers that are neither themselves toxic to the host (e.g., an animal or human), nor degrade (if the polymer degrades) at a rate that produces monomeric or oligomeric subunits or other byproducts that are toxic or are produced at toxic concentrations in the host. In certain embodiments of the present invention, biodegradation generally involves degradation of the polymer in an organism, e.g., into its monomeric subunits, which may be known to be effectively non-toxic. Intermediate oligomeric products resulting from such degradation may have different toxicological properties, however, or biodegradation may involve oxidation or other biochemical reactions that generate molecules other than monomeric subunits of the polymer. Consequently, in certain embodiments, toxicology of a biodegradable polymer intended for in vivo use, such as implantation or injection into a patient, may be determined after one or more toxicity analyses. It is not necessary that any subject composition have a purity of 100% to be deemed biocompatible. Hence, a subject composition may comprise 99%, 98%, 97%, 96%, 95%, 90% 85%, 80%, 75% or even less of biocompatible polymers, e.g., including polymers and other materials and excipients described herein, and still be biocompatible.

To determine whether a polymer or other material is biocompatible, it may be beneficial to conduct a toxicity analysis. Such assays are well known in the art. One example of such an assay may be performed with live carcinoma cells, such as GT3TKB tumor cells, in the following manner: the sample is degraded in 1 M NaOH at 37° C. until complete degradation is observed. The solution is then neutralized with 1 M HCl. About 200 μL of various concentrations of the degraded sample products are placed in 96-well tissue culture plates and seeded with human gastric carcinoma cells (GT3TKB) at 104/well density. The degraded sample products are incubated with the GT3TKB cells for 48 hours. The results of the assay may be plotted as % relative growth vs. concentration of degraded sample in the tissue-culture well. In addition, polymers and compositions of the present invention may also be evaluated by well-known in vivo tests, such as subcutaneous implantations in rats to confirm that they do not cause significant levels of irritation or inflammation at the subcutaneous implantation sites.

When used with respect to a therapeutic agent or other material, the term “sustained release” is art-recognized. For example, a subject composition which releases a substance over time may exhibit sustained release characteristics, in contrast to a bolus type administration in which the entire amount of the substance is made biologically available at one time. For example, in particular embodiments, upon contact with body fluids including blood, spinal fluid, lymph or the like, the HMG-CoA reductase inhibitor/NSAID composition may undergo gradual degradation (e.g., through hydrolysis) with concomitant release of any material incorporated therein, e.g., the HMG-CoA reductase inhibitor and NSAID, for a sustained or extended period (as compared to the release from a bolus). This release may result in prolonged delivery of therapeutically effective amounts of the incorporated therapeutic agent(s).

The term “treating” is art-recognized and includes preventing a disease, disorder or condition from occurring in an animal which may be predisposed to the disease, disorder and/or condition but has not yet been diagnosed as having it; inhibiting the disease, disorder or condition, e.g., impeding its progress; and relieving the disease, disorder, or condition, e.g., causing regression of the disease, disorder and/or condition. Treating the disease or condition includes ameliorating at least one symptom of the particular disease or condition, even if the underlying pathophysiology is not affected, such as treating the pain of a subject by administration of an analgesic agent even though such agent does not treat the cause of the pain.

The phrase “pharmaceutically acceptable” is art-recognized. In certain embodiments, the term includes compositions, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” is art-recognized, and includes, for example, pharmaceutically acceptable materials, compositions or vehicles, such as a liquid or solid filler, diluent, solvent or encapsulating material involved in carrying or transporting any subject composition, from one organ, or portion of the body, to another organ, or portion of the body. Each carrier must be “acceptable” in the sense of being compatible with the other ingredients of a subject composition and not injurious to the patient. In certain embodiments, a pharmaceutically acceptable carrier is non-pyrogenic. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) cocoa butter and suppository waxes; (9) oils, such as peanut oil, cottonseed oil, sunflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical compositions.

The term “pharmaceutically acceptable salts” is art-recognized, and includes relatively non-toxic, inorganic and organic acid addition salts of compositions, including without limitation, analgesic agents, therapeutic agents, other materials and the like. Examples of pharmaceutically acceptable salts include those derived from mineral acids, such as hydrochloric acid and sulfuric acid, and those derived from organic acids, such as ethanesulfonic acid, benzenesulfonic acid, p-toluenesulfonic acid, and the like. Examples of suitable inorganic bases for the formation of salts include the hydroxides, carbonates, and bicarbonates of ammonia, sodium, lithium, potassium, calcium, magnesium, aluminum, zinc and the like. Salts may also be formed with suitable organic bases, including those that are non-toxic and strong enough to form such salts. For purposes of illustration, the class of such organic bases may include mono-, di-, and trialkylamines, such as methylamine, dimethylamine, and triethylamine; mono-, di- or trihydroxyalkylamines such as mono-, di-, and triethanolamine; amino acids, such as arginine and lysine; guanidine; N-methylglucosamine; N-methylglucamine; L-glutamine; N-methylpiperazine; morpholine; ethylenediamine; N-benzylphenethylamine; (trihydroxymethyl)aminoethane; and the like. See, for example, J. Pharm. Sci. 66: 1-19 (1977).

A “patient,” “subject,” or “host” to be treated by the subject method may mean either a human or non-human animal, such as primates, mammals, and vertebrates.

The term “prophylactic or therapeutic” treatment is art-recognized and includes administration to the host of one or more of the subject compositions. If it is administered prior to clinical manifestation of the unwanted condition (e.g., disease or other unwanted state of the host animal) then the treatment is prophylactic, i.e., it protects the host against developing the unwanted condition, whereas if it is administered after manifestation of the unwanted condition, the treatment is therapeutic (i.e., it is intended to diminish, ameliorate, or stabilize the existing unwanted condition or side effects thereof).

The term “preventing” is art-recognized, and when used in relation to a condition, such as a local recurrence (e.g., pain), a disease such as cancer, a syndrome complex such as heart failure or any other medical condition, is well understood in the art, and includes administration of a composition which reduces the frequency of or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount. Prevention of an infection includes, for example, reducing the number of diagnoses of the infection in a treated population versus an untreated control population, and/or delaying the onset of symptoms of the infection in a treated population versus an untreated control population. Prevention of pain includes, for example, reducing the magnitude of, or alternatively delaying, pain sensations experienced by subjects in a treated population versus an untreated control population.

The phrases “systemic administration,” “administered systemically,” “peripheral administration” and “administered peripherally” are art-recognized, and include the administration of a subject composition, therapeutic or other material at a site remote from the disease being treated. Administration of an agent directly into, onto, or in the vicinity of a lesion of the disease being treated, even if the agent is subsequently distributed systemically, may be termed “local” or “topical” or “regional” administration, particularly where the agent does not reach therapeutically effective levels systemically, e.g., has a higher local concentration.

The phrase “therapeutically effective amount” is an art-recognized term. In certain embodiments, the term refers to an amount of the therapeutic agent that, when incorporated into a polymer of the present invention, produces some desired effect at a reasonable benefit/risk ratio applicable to any medical treatment. In certain embodiments, the term refers to that amount necessary or sufficient to eliminate or reduce sensations of pain for a period of time. The effective amount may vary depending on such factors as the disease or condition being treated, the particular targeted constructs being administered, the size of the subject, or the severity of the disease or condition. One of ordinary skill in the art may empirically determine the effective amount of a particular compound without necessitating undue experimentation.

The term “ED₅₀” is art-recognized. In certain embodiments, ED₅₀ means the dose of a drug that produces 50% of its maximum response or effect, or, alternatively, the dose that produces a pre-determined response in 50% of test subjects or preparations.

### Sustained-Release Compositions

In certain embodiments, compositions of the invention comprise at least one HMG-CoA reductase inhibitor and at least one NSAID which account for 90% or more by weight of the composition. In certain embodiments, the NSAID of the composition is selected from naproxen, diclofenac, celecoxib, sulindac, diflunisal, piroxicam, indomethacin, etodolac, meloxicam, ibuprofen, ketoprofen, mefenamic acid, nabumetone, tolmetin, ketorolac tromethamine, choline magnesium trisalicylate, rofecoxib, aspirin, and paracetamol or a pharmaceutically acceptable salt thereof In certain embodiments the composition comprises more than one NSAID, such as two or three NSAIDs. In certain embodiments, the NSAID is selected from cyclooxygenase-2 inhibitors (COX-2 inhibitors) such as celecoxib, etoricoxib, lumiracoxib, parecoxib, rofecoxib, and vladecoxib. In certain embodiments, the composition comprises more than one COX-2 inhibitor. In preferred embodiments, the NSAID is celecoxib.

In certain embodiments, the HMG CoA reductase inhibitor of the composition is selected from cerivastatin, fluvastatin, atorvastatin, lovastatin, pravastatin, and simvastatin or a pharmaceutically acceptable salt thereof. In preferred embodiments, the HMG-CoA reductase inhibitor is simvastatin. In particular embodiments, the composition comprises simvastatin and celecoxib.

In certain embodiments, the composition comprises greater than 90% by weight of celecoxib and an HMG-CoA reductase inhibitor, e.g., simvastatin. In certain embodiments the composition comprises greater than 90% by weight of simvastatin and an NSAID, e.g., celecoxib. In particular embodiments, the compositions of the invention comprise greater than 90% by weight of simvastatin and celecoxib, such as greater than 95%, such as greater than 99%.

In certain embodiments, HMG-CoA reductase inhibitor/NSAID compositions of the invention are essentially free of excipients. As used herein, the term “essentially free of” excipients, such as antiadherents, binders, coatings, disintegrants, fillers, diluents, flavours, colours, glidants, lubricants, preservatives, sorbents and sweeteners, is used to indicate that the HMG-CoA inhibitor/NSAID compositions of the invention are substantially devoid of excipients. Expressed in terms of purity, essentially free means that the amount of excipients in a composition does not exceed 10%, such as below about 5%, such as below about 1%, such as below about 0.5%, wherein the percentages are by weight.

In certain embodiments, the composition comprises a mole ratio of simvastatin to celecoxib selected from 10:1 to 1:10. In certain embodiments, the mole ratio of simvastatin to celecoxib is selected from about 5:1 to 1:5, such as from about 5:1 to 2:1, such as about 3:1, such as about 2.75:1, such as about 2.5:1, such as about 2.25:1 such as about 2:1, such as about 1.75:1, such as about 1.5:1 such as about 1.25:1, such as about 1:1, such as about 1:1.25, such as about 1:1.5, such as about 1:1.75, such as about 1:2, such as about 1:2.25, such as about 1:2.5, such as about 1:2.75, such as about 1:3. In particular embodiments, simvastatin is in excess to celecoxib with a mole ratio of about 2.75:1.

In certain embodiments, the compositions of the invention are micro- or macroparticles. In certain embodiments, the particles of the compositions may appear to have a geometrically definable shape such as a sphere or a cube or may be in an amorphous form. In any case, the size of an individual particle can be determined by the largest diameter or cross-sectional dimension of the particle. In certain embodiments, the microparticles range in diameter from 0.1 ∞m to 100 ∞m, such as from 1 to 50 ∞m, such as from 5 to 25 ∞m. In certain embodiments, the average particle diameter is about 10 ∞m, about 15 ∞m, about 20 ∞m, about 25 μm, about 30 ∞m, about 35 ∞m, about 40 ∞m, about 45 ∞m, about 50 ∞m, or about 25 ∞m.

In certain embodiments, the composition may include one or more additional components such as pharmaceutically acceptable carriers or an additional therapeutic agent. In certain embodiments, the composition comprises a NSAID, and a HMG-CoA reductase inhibitor and a biocompatible polymer, such as N-methyl-2-pyrrolidone (NMP). Biocompatible polymers are known in the art and exemplary polymers are presented herein. In certain embodiments, the additional components of the composition account for 10% or less of the weight of the compositions. In certain embodiments, the composition comprises less than about 10% of additional agents. such as less than about 9%, such as less than about 8%, such as less than about 7%, such as less than about 6%, such as less than about 5%, such as less than about 4%, such as less than about 3%, such as less than about 2%, such as less than about 1% of agents aside from an NSAID, e.g., celecoxib, and an HMG-CoA reductase inhibitor, e.g., simvastatin.

### Preparation of Sustained-Release Compositions

In certain embodiments, drug delivery compositions of the invention are prepared by the method comprising: a. providing a solution comprising a water-miscible solvent, e.g., an organic solvent; a HMG CoA inhibitor, e.g., simvastatin, and a NSAID, e.g., celecoxib, and b. contacting the solution with an aqueous medium, e.g., blood, cerebrospinal fluid, Hank's solution or Ringer's solution, under conditions that induce precipitation of a solid comprising simvastatin and celecoxib.

Solutions of step “a” of the method may be prepared through known methods of preparing solutions or obtained through, for example, a commercial vendor. In certain embodiments, the solutions may be prepared by dissolving a NSAID, e.g., celecoxib, and an HMG CoA inhibitor, e.g., simvastatin, in a water miscible solvent, e.g., such as a polar aprotic organic solvent. Exemplary solvents include dimethyl sulfoxide (DMSO), N,N-dimethylformamide (DMF), dioxane, hexamethylphosphorous triamide (HMPT), N-methyl-2-pyrrolidone (NMP), and tetrahydrofuran. Solutions may include additional components such as excipients, diluents or solvents such as organic solvents. In preferred embodiments, the solution comprises N-methyl-2-pyrrolidone.

In certain embodiments, the concentration of the solution formed has a molar ratio of simvastatin to celecoxib from 10:1 to 1:10. In certain embodiments, the molar ratio of simvastatin to celecoxib is selected from about 5:1 to 1:5, such as from 5:1 to 2:1, such as about 3:1, such as about 2.75:1, such as about 2.5:1, such as about 2.25:1, such as about 2:1, such as about 1.75:1, such as about 1.5:1, such as about 1.25:1, such as about 1:1, such as about 1:1.25, such as about 1:1.5, such as about 1:1.75, such as about 1:2, such as about 1:2.25, such as about 1:2.5, such as about 1:2.75, such as about 1:3. In particular embodiments, simvastatin is in excess to celecoxib with a molar ratio of about 2.75:1.

In certain embodiments, contacting the solution of step “a” with the aqueous medium in step “b” comprises adding the solution of step “a” to the aqueous medium of step “b.” In certain embodiments, the solution is added in aliquots to the aqueous medium. In particular embodiments, one or more aliquots of the solution, e.g., volumes of from 5 to 200 μL, such as about 50 μL, such as about 10 μL, are added to the aqueous medium, such as by pipette or syringe. In certain embodiments, contacting the solution of step “a” with the aqueous medium of step “b” occurs though introduction of the solution of step “a” into a patient's body, e.g., by injection of the solution into the body of a patient. In certain embodiments, the aqueous medium is added to the solution.

In certain embodiments, the method of preparing the drug delivery composition comprises contacting the solution of step “a” with the aqueous medium in step “b” such as an aqueous medium within a human body, e.g., blood, serum. In certain such embodiments, the solution may be injected at a location in the body in need of the therapeutic treatment, e.g., a tumor in the case of cancer.

In certain embodiments, the method of preparing the drug delivery composition comprises contacting the solution of step “a” with the aqueous medium in step “b” in which the aqueous medium is selected from water, a solution or a buffer, e.g., HEPES solution, Ringer's solution, etc. In certain such embodiments, the method may further comprise the additional step of isolating the precipitated particles that form when a solution of step “a” is contacted with the aqueous medium of step “b”. The precipitate may be isolated by any suitable method for separating a solid particulate from a liquid. Exemplary approaches to separating the precipitate from the aqueous medium include pelleting the product through centrifugation and decanting the liquid from the pellet, or filtration of the particles though any suitable method for filtering particles from a liquid phase. In particular embodiments, components of the solution and/or the aqueous medium may be modified to obtain particles with desired sustained-release characteristics, e.g., longer or shorter sustained release profiles.

In other embodiments, the precipitate is not isolated from the aqueous medium. In such embodiments, the mixture of step “b” may be used for administration to a patient with or without modification of the mixture. For example, the mixture may be modified to decrease the amount of liquid, sterilize the mixture, or add excipients or diluents. Alternately the mixture may be administered to a patient without any additional components.

### Pharmaceutical Formulations

The sustained-release compositions of the invention may be administered to a patient or they may be modified to form pharmaceutical formulations through methods such as those known in the art for forming pharmaceutical formulations. Exemplary modifications include the sterilization of the compositions, the addition of coatings, binders, or excipients. The compositions of the invention may be formed into pharmaceutical formulations using one or more physiologically or pharmaceutically acceptable carriers or excipients. For example, compositions of HMG-CoA reductase inhibitor and an NSAID may be formed into pharmaceutical formulations for administration by, for example, injection (e.g., SubQ, IM, IP), inhalation or insufflation (either through the mouth or the nose) or oral, buccal, sublingual, transdermal, nasal, parenteral or rectal administration. In one embodiment, a HMG-CoA reductase inhibitor/NSAID formulation may be administered locally, at the site where the target cells are present, i.e., in a specific tissue, organ, or fluid (e.g., blood, cerebrospinal fluid, etc.). In particular embodiments, formulations of the invention are administered within or in proximity to a tumor, e.g., for treating cancer.

HMG-CoA reductase inhibitor/NSAID compositions can be formed into pharmaceutical formulations for a variety of modes of administration, including systemic and topical or localized administration. Techniques and formulations generally may be found in Remington's Pharmaceutical Sciences, Meade Publishing Co., Easton, Pa. For parenteral administration, injection is preferred, including intramuscular, intravenous, intraperitoneal, and subcutaneous. For injection, the compositions can be formulated in liquid suspensions, preferably in physiologically compatible buffers such as Hank's solution or Ringer's solution. In addition, the compositions may be formulated in solid form and suspended immediately prior to use. Lyophilized forms are also included.

For administration by inhalation (e.g., pulmonary delivery), HMG-CoA reductase inhibitor/NSAID formulations may be conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin, for use in an inhaler or insufflator may be formulated containing a powder mix of the compound and a suitable powder base such as lactose or starch.

HMG-CoA reductase inhibitor/NSAID formulations may be prepared for parenteral administration by injection, e.g., by bolus injection or continuous infusion. Formulations for injection may be presented in unit dosage form, e.g., in ampoules or in multi-dose containers, with an added preservative. The formulations may take such forms as suspensions, solutions or emulsions in oily or aqueous vehicles, and may contain formulatory agents such as suspending, stabilizing and/or dispersing agents. Alternatively, the HMG-CoA reductase inhibitor/NSAID formulations may be in particulate form for constitution with a suitable vehicle, e.g., sterile pyrogen-free water, before use.

HMG-CoA reductase inhibitor/NSAID compositions may also be formed in rectal formulations such as suppositories or retention enemas, e.g., containing conventional suppository bases such as cocoa butter or other glycerides.

HMG-CoA reductase inhibitor/NSAID compositions may also be formulated as a depot preparation. Such long acting formulations may be administered by implantation (for example subcutaneously or intramuscularly) or by intramuscular injection. Thus, for example, HMG-CoA reductase inhibitor/NSAID compositions may be formulated with suitable polymeric or hydrophobic materials (for example as an emulsion in an acceptable oil) or ion exchange resins.

In certain embodiments, the compositions described herein can be formulated for delivery to the central nervous system. Conventional approaches for drug delivery to the CNS include: neurosurgical strategies (e.g., intracerebral injection or intracerebroventricular infusion); pharmacological strategies designed to increase the lipid solubility of an agent (e.g., conjugation of water-soluble agents to lipid or cholesterol carriers); and the transitory disruption of the integrity of the BBB by hyperosmotic disruption (resulting from the infusion of a mannitol solution into the carotid artery or the use of a biologically active agent such as an angiotensin peptide).

Formulations may be colorless, odorless ointments, lotions, creams, microemulsions and gels.

HMG-CoA reductase inhibitor/NSAID compositions may be incorporated into ointments, which generally are semisolid preparations which are typically based on petrolatum or other petroleum derivatives. The specific ointment base to be used, as will be appreciated by those skilled in the art, is one that will provide for optimum drug delivery, and, preferably, will provide for other desired characteristics as well, e.g., emolliency or the like. As with other carriers or vehicles, an ointment base should be inert, stable, nonirritating and nonsensitizing.

HMG-CoA reductase inhibitor/NSAID compositions may be incorporated into gel formulations, which generally are semisolid systems consisting of either suspensions made up of small inorganic particles (two-phase systems) or large organic molecules distributed substantially uniformly throughout a carrier liquid (single phase gels). Although gels commonly employ aqueous carrier liquid, alcohols and oils can be used as the carrier liquid as well.

Other active agents may also be included in formulations, e.g., other anti-inflammatory agents, analgesics, antimicrobial agents, antifungal agents, antibiotics, vitamins, antioxidants, and sunblock agents commonly found in sunscreen formulations including, but not limited to, anthranilates, benzophenones (particularly benzophenone-3), camphor derivatives, cinnamates (e.g., octyl methoxycinnamate), dibenzoyl methanes (e.g., butyl methoxydibenzoyl methane), p-aminobenzoic acid (PABA) and derivatives thereof, and salicylates (e.g., octyl salicylate).

HMG-CoA reductase inhibitor/NSAID formulations described herein may be stored in an oxygen free environment.

Data obtained from cell culture assays and animal studies can be used in formulating a range of dosage for use in humans. The dosage of such compounds may lie within a range of circulating concentrations that include the ED₅₀ with little or no toxicity. The dosage may vary within this range depending upon the dosage form employed and the route of administration utilized. The therapeutically effective dose can be estimated initially from cell culture assays. A dose may be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

### Indications

The HMG-CoA reductase inhibitor/NSAID compositions and formulations of the present invention are useful in treating or preventing neurological disorders including, but not limited to, Alzheimer's disease amyotrophic lateral sclerosis, epilepsy, Huntington's Disease, Parkinson's Disease, stroke, spinal cord injury, traumatic brain injury, Lewy body dementia, multiple sclerosis, Pick's disease, Niewmann-Pick disease, amyloid angiopathy, cerebral amyloid angiopathy, systemic amyloidosis, hereditary cerebral hemorrhage with amyloidosis of the Dutch type, inclusion body myositis, mild cognitive impairment and Down's syndrome. The HMG-CoA reductase inhibitor/NSAID compositions and formulations are also useful in treating complications due to stroke, head trauma, or spinal injury, or other injuries to the brain, peripheral nervous, central nervous, or neuromuscular system, and in the preparation of medicaments therefore.

HMG-CoA reductase inhibitor/NSAID compositions and formulations may be used for treating or preventing cancer. Exemplary cancers that may be treated using HMG-CoA reductase inhibitor/NSAID formulations include but are not limited to those of the brain and kidney; hormone-dependent cancers including breast, prostate, testicular, and ovarian cancers; lymphomas, and leukemias. In cancers associated with solid tumors, an HMG-CoA reductase inhibitor/NSAID formulations may be administered directly into the tumor. Cancer of blood cells, e.g., leukemia, can be treated by administering an HMG-CoA reductase inhibitor/NSAID formulation into the blood stream or into the bone marrow.

### EXEMPLIFICATION

### Preparation of Stock Solution of Celecoxib and Simvastatin

50 mg of celecoxib and 150 mg of simvastatin were dissolved in 250 μL of N-methyl-2-pyrrolidone. The solution is clear.

### Test of Release Rates of Celecoxib and Simvastatin

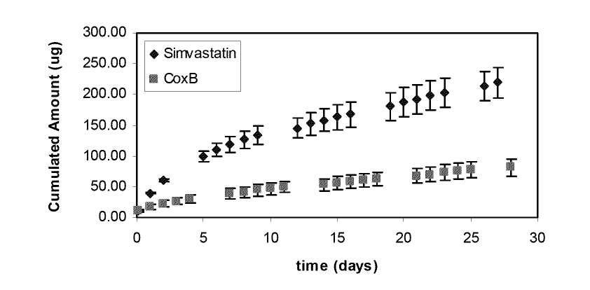

In a glass vial, 10 μL celecoxib/simvastatin stock solution was added to 2.0 mL phosphate buffer (0.1 M, pH 7.4) and a round shaped solid precipitate was formed in about 30 minutes. After 2 hours the buffer was removed and 2.0 mL of fresh buffer added to the glass vial. The vial was placed in a water bath at 37° C. for multiple days. Every 24 hours a sample was removed to assay the celecoxib and simvastatin content of the buffer (FIG. 1). At those 24 hour intervals, the buffer was removed and replaced with fresh buffer each time.

We claim:

1. A drug delivery composition comprising celecoxib and simvastatin, wherein greater than 90% of the weight of the formulation is celecoxib and simvastatin.

2. The drug delivery composition of claim 1, wherein the mole ratio of simvastatin to celecoxib is selected from 3:1 to 1:3.

3. The drug delivery composition of claim 2, wherein the mole ratio of simvastatin to celecoxib is about 2:1.

4. The drug delivery composition of claim 2, wherein the mole ratio of celecoxib to simvastatin is about 2:1.

5. The drug delivery composition of claim 2, wherein the mole ratio of simvastatin to celecoxib is about 2.75:1.

6. The drug delivery composition of claim 1, wherein greater than 95% of the weight of the formulation is celecoxib and simvastatin.

7. The drug delivery composition of claim 6, wherein greater than 99% of the weight of the formulation is celecoxib and simvastatin.

8. The drug delivery composition of claim 1, wherein the formulation is essentially free of excipients and diluents.

9. The drug delivery composition of claim 1, wherein the formulation provides sustained release of celecoxib in a biological medium.

10. The drug delivery composition of claim 1, wherein the formulation provides sustained release of simvastatin in a biological medium.

11. A method of preparing a drug delivery composition comprising:
a. providing a solution comprising a water-miscible solvent, simvastatin, and celecoxib, and
b. contacting the solution with an aqueous medium under conditions that induce precipitation of a solid comprising simvastatin and celecoxib.

12. The method of claim 11, wherein the water-miscible solvent is an organic solvent.

13. The method of claim 12, wherein the organic solvent is selected from N-methyl 2-pyrrolidone (NMP), dimethylsulfoxide (DMSO), N,N-dimethylformamide (DMF), and N,N-dimethylacetamide.

14. The method of claim 13, wherein the solvent is N-methyl 2-pyrrolidone.

15. The method of claim 11, wherein the mole ratio of simvastatin to celecoxib is about 2:1.

16. The method of claim 11, wherein the mole ratio of celecoxib to simvastatin is about 2:1.

17. The method of claim 11, wherein the mole ratio of simvastatin to celecoxib is about 2.75:1.

18. The method of claim 11, wherein the solution is contacted with the aqueous medium by addition of one or more aliquots of the solution to the aqueous medium.

19. The method of claim 18, wherein each aliquot has a volume from 5 to 200 μL.

20. The method of claim 19, wherein each aliquot has a volume from 10 to 100 μL.

21. The method of claim 11, further comprising isolating the solid from the aqueous medium.

22. A method for treating or preventing a neurodegenerative disorder, comprising administering a drug delivery formulation of claim 1 to a patient in need thereof.

23. The method of claim 22, wherein the neurodegenerative disorder is selected from Alzheimer's disease (AD), Parkinson's disease (PD), Huntington disease (HD), amyotrophic lateral sclerosis (ALS; Lou Gehrig's disease), diffuse Lewy body disease, chorea-acanthocytosis, primary lateral sclerosis, Multiple Sclerosis (MS) and Friedreich's ataxia.

24. A method for treating cancer, comprising administering a drug delivery formulation of claim 1 to a patient in need thereof.

25. The method of claim 22, wherein the drug delivery formulation provides sustained release of celecoxib and simvastatin over a period of greater than 20 days.
